(19) United States
(12) Patent Application Publication
(10) Pub. No.: US 2023/0190654 A1
(43) Pub. Date: Jun. 22, 2023

(54) DEXAMETHASONE-LOADED MACROPHAGE-DERIVED MICROVESICLE AS WELL AS PREPARATION METHOD AND APPLICATION THEREOF

(71) Applicant: SOUTHEAST UNIVERSITY, Nanjing (CN)

(72) Inventors: Bicheng LIU, Nanjing (CN); Taotao TANG, Nanjing (CN); Linli LV, Nanjing (CN)

(21) Appl. No.: 16/462,172

(22) PCT Filed: Sep. 21, 2018
(86) PCT No.: PCT/CN2018/106859

§ 371 Date: May 17, 2019

(30) Foreign Application Priority Data
May 2, 2018 (CN) 201810409507.0

(51) Int. Cl. A61K 9/127; A61K 31/573; A61K 9/50; A61P 13/12; A61P 29/00; A61P 37/06

(52) U.S. Cl. CPC A61K 9/1278; A61K 31/573; A61K 9/5068; A61P 13/12; A61P 29/00; A61P 37/06

(57) ABSTRACT

It discloses a dexamethasone-loaded macrophage-derived microvesicle as well as a preparation method and application thereof. The dexamethasone-loaded macrophage-derived microvesicle is formed by entrapping dexamethasone with a microvesicle derived from a murine macrophage cell line 264.7 cell. The dexamethasone-loaded macrophage-derived microvesicle of the present invention can be taken up by an injured cell more effectively, and fulfills the aim of ameliorating the kidney inflammation by inhibiting the activation of a proinflammatory signal pathway and infiltration of inflammatory cells. Meanwhile, the preparation method of the present invention is simple, convenient and efficient, and the prepared microvesicle is derived from the RAW 264.7 cells which are sufficient and widespread, and can be produced in large-scale.

### TECHNICAL FIELD

The present invention belongs to the field of biomedicine, and relates to a microvesicle-based drug delivery system, and more particularly, to a preparation method of a dexamethasone-loaded macrophage-derived microvesicle and an application thereof in anti-inflammatory treatment for kidney diseases.

### BACKGROUND

Glucocorticoids such as dexamethasone are widely used in the treatment of inflammatory and immune diseases due to remarkable anti-inflammatory and immunosuppressive effects, and are mainly used in the treatment of acute glomerulonephritis, nephrotic syndrome, IgA nephropathy, etc. However, long-term massive use of glucocorticoids may lead to serious adverse reaction and glucocorticoid resistance, which seriously hinders clinical use. Therefore, the exploration of a method to reduce adverse reaction of glucocorticoid and improve glucocorticoid sensitivity has become a research hotspot.

The microvesicle is an extracellular vesicle with a diameter of about 100 to 1000 nm secreted by various living cells, which contains abundant proteins, lipids, nucleic acids and membrane receptors from parent cells, and plays a vital role in a variety of physiological and pathological processes. In recent years, it has been found that the extracellular vesicle, as a natural and stable nano-scale membrane vesicle, can penetrate biological barrier, protect the content thereof from degradation, and be efficiently taken up by recipient cells. Therefore, it can be used as a carrier of gene and drug to participate in the treatment of diseases. Compared with other commonly used treatment carriers such as virus and liposome, the extracellular vesicle is also characterized by low immunogenicity and non-cytotoxicity.

At present, patent document CN102596177A, published on Jul. 18, 2012, discloses microvesicles derived from nucleated mammalian cells, the microvesicles are smaller than the nucleated cells and can be used to deliver therapeutic substances or diagnostic substances to specific tissues or specific cells. More specifically, the invention relates to the microbubbles derived from monocytes, macrophages, dendritic cells, stem cells and the like, the microbubbles can be used to deliver specific therapeutic substances or diagnostic substances for treating and/or diagnosing tissues related to cancer, vascular disease, inflammation and the like. However, there is no report in the prior art that a macrophage microvesicle is used as a dexamethasone carrier to realize anti-inflammatory treatment for kidney diseases.

### SUMMARY

Object of the invention: regarding the problems of the prior art, the present invention provides a dexamethasone (DEX)-loaded macrophage-derived microvesicle, which can be taken up by an injured cell more effectively, and fulfills the purpose of ameliorating the kidney inflammation by inhibiting the activation of a proinflammatory signal pathway and infiltration of inflammatory cells.

The present invention further provides a preparation method and application of the dexamethasone-loaded macrophage-derived microvesicle.

Technical solution: in order to achieve the object above, the dexamethasone-loaded macrophage-derived microvesicle of the present invention is formed by entrapping dexamethasone with a microvesicle derived from a murine macrophage cell line RAW264.7 cell. The dexamethasone-loaded macrophage-derived microvesicle of the present invention is named Mφ-MP-DEX, the microbubble is derived from the RAW 264.7 cell of the mouse macrophage system, and dexamethasone (DEX) is entrapped inside the microvesicle.

The preparation method of the dexamethasone-loaded macrophage-derived microvesicle of the present invention comprises the following steps:

(1) culturing a murine macrophage cell line RAW264.7 cell;

(2) processing the RAW 264.7 cell with dexamethasone; and

(3) collecting medium supernatant after the cell is processed with dexamethasone in step (2), and collecting a microvesicle by differential centrifugation to obtain the dexamethasone-loaded macrophage-derived microvesicle.

A specific process of culturing the RAW 264.7 cell of the mouse macrophage system in step (1) is: culturing the RAW 264.7 cell in a RIPM 1640 medium containing 10% fetal bovine serum, placing in an 37° C. incubator with saturated humidity and 5% CO2, and conducting the processing in step (2) when cell reaches 70% to 80% confluence.

The fetal bovine serum (FBS) for culturing the RAW 264.7 cell in step (1) is a fetal bovine serum of which a microvesicle is removed.

A specific process of processing the RAW 264.7 cell with dexamethasone in step (2) is: when the RAW 264.7 cell cultured in step (1) reaches 70% to 80% confluence, washing the cell with a RIPM 1640 medium not containing fetal bovine serum, and then replacing the medium by the RIPM 1640 medium without FBS, and then adding dexamethasone to process. Dexamethasone is added to make the final concentration be 30 μmol/L to process for 16 h.

A specific process of step (3) is: collecting the medium supernatant after the cell is processed with dexamethasone in step (2), conducting low speed centrifugation firstly to obtain the supernatant, then conducting high speed centrifugation to discard the supernatant, adding PBS for resuspension washing, and then conducting high speed centrifugation to obtain a sediment which is the dexamethasone-loaded macrophage-derived microvesicle. The microvesicle is re-suspended with sterile PBS or sterile physiological saline to be stored at −80° C.

The application of the dexamethasone-loaded macrophage-derived microvesicle of the present invention in preparing a drug or preparation for treating kidney diseases is also disclosed.

Further, the dexamethasone-loaded macrophage-derived microvesicle can be better taken up by an injured cell and inhibit the activation of a proinflammatory signal pathway and infiltration of inflammatory cells, so as to achieve the aim of treating kidney inflammation and realize the application in preparing the drug or preparation for treating kidney diseases.

The application of the dexamethasone-loaded macrophage-derived microvesicle of the present invention in preparing an anti-inflammatory or immunosuppressive drug or preparation is also disclosed.

An average diameter of the Mφ-MP-DEX of the present invention is about 140.7±4.8 nm, a morphology observed under a transmission electron microscope conforms to the characteristics of the microvesicle, and DEX entrapped in the microvesicle can be detected by high performance liquid chromatography.

Through cell experiment, the present invention proves that the Mφ-MP-DEX can be taken up by murine glomerular endothelial cells (GECs) and has inflammatory dependence, that is, after the intervention of lipopolysaccharide (LPS), the uptake of the microvesicle by the GECs can be increased, and meanwhile, the inflammatory reaction caused by LPS is obviously improved.

Further, the present invention proves for the first time that under the same drug concentration, the anti-inflammatory effect of the dexamethasone-loaded macrophage-derived microvesicle (Mφ-MP-DEX) is stronger than that of direct administration of DEX, which shows more effective inhibition to the activation of NF-κB signal pathway and reduction of pro-inflammatory factors.

Since kidneys of LPS-induced sepsis model have significant inflammatory reaction, the present invention constructs this murine model to evaluate the therapeutic efficacy of Mφ-MP-DEX. Mice are injected intravenously with Mφ-MP-DEX or bare DEX with equimolar doses through tail vein. Mφ-MP-DEX exhibits a superior capacity to suppress renal inflammation when compared to bare DEX treatment.

According to the present invention, the animal experiment proves for the first time that My-MP-DEX inhibits the expression of the pro-inflammatory factors and NF-κB more effectively than that of direct administration of DEX, and reduces the interstitial infiltration of macrophage.

Compared with the prior art, the present invention has the following advantages.

1. According to the present invention, the macrophage-derived microvesicle is utilized to entrap DEX, and the obtained Mφ-MP-DEX has a stronger anti-inflammatory effect than that of direct administration of DEX and a better treatment effect on kidney inflammation. Therefore, the Mφ-MP-DEX prepared by the present invention can be applied to preparing the drug or preparation for treating kidney diseases, and has a very considerable application prospect in clinical treatment of kidney diseases.

2. The Mφ-MP-DEX prepared by the present invention has a stronger anti-inflammatory effect, so that the dosage of DEX can be reduced, and adverse reaction caused by glucocorticoid can be obviously improved.

3. The preparation method of the present invention is simple, convenient and efficient, and the prepared microvesicle is derived from the RAW 264.7 cells which are sufficient and widespread, and can be produced in large-scale.

4. According to the present invention, since DEX has extensive anti-inflammatory and immunosuppressive effects, the prepared dexamethasone-loaded macrophage-derived microvesicle also has a good application prospect in other inflammatory and immune diseases, including rheumatoid arthritis, multiple sclerosis, systemic lupus erythematosus, inflammatory bowel disease, asthma and other diseases, and the macrophage-derived microvesicle of the present invention can also be applied to the preparation of anti-inflammatory or immunosuppressive drug or preparation.

### BRIEF DESCRIPTION OF THE DRAWINGS

FIG. 1 is an identification diagram of a dexamethasone-loaded macrophage-derived microvesicle: (A) detection of macrophage marker proteins F4/80 and CD68 by Western blot; (B) observation of morphology of Mφ-MP-DEX by a transmission electron microscope with a scale of 100 nm; (C) particle size distribution diagram of Mφ-MP-DEX; and (D) concentration determination of dexamethasone in Mφ-MP-DEX;

FIG. 2 is an analysis diagram of taking up Mφ-MP-DEX by a glomerular endothelial cell: (A) average fluorescence intensity after Mφ-MP-DEX is taken in by the glomerular endothelial cell; and (B) number of Mφ-MP-DEXs taken in by the glomerular endothelial cell, with an amplification times of 1000;

FIG. 3 is a diagram of an anti-inflammatory effect of Mφ-MP-DEX: (A) mRNA level of the pro-inflammatory factor;

FIG. 4 is a comparison diagram of anti-inflammatory effects of Mφ-MP-DEX and DEX: (A) the mRNA level of the pro-inflammatory factor; and (B) protein levels of NF-κB p65 and p-p65;

FIG. 5 is a comparison diagram of treatment effects of Mφ-MP-DEX and DEX in a mouse sepsis model: (A) mRNA level of the pro-inflammatory factor; (B) protein levels of NF-κB p65 and p-p65; and (C) interstitial macrophage staining with an amplification times of 200.

### DETAILED DESCRIPTION

The present invention is further described hereinafter with reference to the drawings and the embodiments.

### First Embodiment

Generation, Identification and Drug Concentration Determination of Dexamethasone-Loaded Macrophage-Derived Microvesicle (Mφ-MP-DEX)

1. Generation of Mφ-MP-DEX

The murine macrophage cell line RAW 264.7 cells (purchased from a cell bank of Chinese Academy of Sciences) were cultured in a RIPM 1640 medium (Gibco of America) containing 10% fetal bovine serum (Gibco of America), wherein a microvesicle of the used fetal bovine serum was removed by centrifugation. When the RAW 264.7 cells in a culture flask reached 70% to 80% confluence, the cells were washed twice with the RIPM 1640 medium, the medium was replaced with the RIPM 1640 medium without FBS, and the DEX (Sigma of America) was added to make the final concentration be 30 μmol/L to process for 16 h. After processing for 16 h, a medium supernatant was collected aseptically in a 50 ml sterile centrifuge tube and centrifuged at 4° C. and 2000×g for 20 min to remove dead cells and large debris; the supernatant was carefully transferred to a new sterile centrifuge tube and centrifuged at 4° C. and 10000×g for 2 min to remove dead cell and small debris; and the supernatant was carefully transferred to a new 50 ml sterile centrifuge tube and centrifuged at 4° C. and 16500×g for 30 min, the supernatant was carefully discarded, then sterile PBS (pH 7.4) was add for resuspension washing once and centrifuged at 4° C. and 16500×g for 30 min to obtain a precipitate, which was the macrophage-derived microvesicle (Mφ-MP-DEX), and the microvesicle was resuspended with sterile PBS or sterile physiological saline and stored at −80° C.

2. Identification of Mφ-MP-DEX

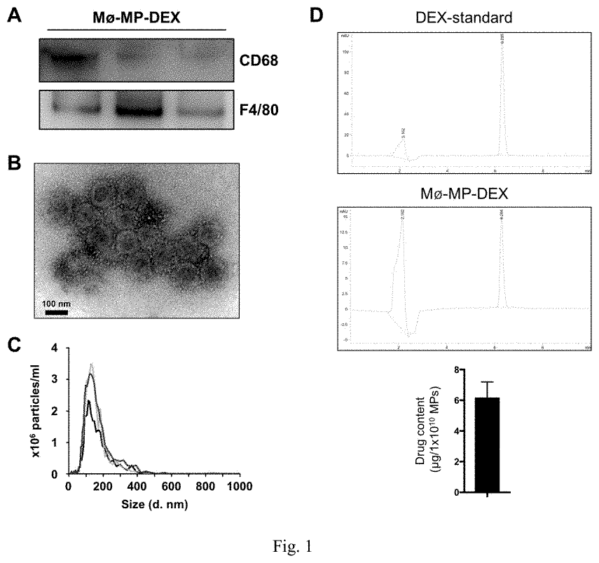

Results of detecting marker proteins on the surface of the prepared microvesicle by Western blot were shown in FIG. 1A, and two membrane proteins of macrophage (including CD68 and F4/80) were identified in Mφ-MP-DEX, proved that the microvesicle was derived from the macrophage.

A morphology of Mφ-MP-DEX was observed by a transmission electron microscope: My-MP-DEX was fully and evenly blown with a pipette gun, 10 μL liquid was sucked and dropped onto a 200-mesh sample-carrying copper grid, and stood at a room temperature for 1 h, then excess liquid was carefully sucked off with filter paper, and the sample was imaged by the transmission electron microscope after drying. The results were shown in FIG. 1B, a circular double-layer membrane structure with a diameter of about 150 nm could be seen, which proved that the prepared Mφ-MP-DEX conformed to the characteristics of the microvesicle.

Particle size detection of microvesicle: the microvesicle was sent to Shanghai Xiaopeng Biotechnology Co., Ltd. to detect particle size distribution using German PMX nanoparticle tracking analyzer Zetaview. The results were as shown in FIG. 1C, and an average diameter of the microvesicle was 140.7±4.8 nm.

3. Drug Concentration Determination of Mφ-MP-DEX

The concentration of DEX in the microvesicle was detected by HPLC method: firstly, the concentration of the Mφ-MP-DEX was determined by the German PMX nanoparticle tracking analyzer, and then an appropriate amount (in a detection range) of microvesicle resuspension (a total number of the microvesicles was about 5×10⁹) was taken for detection. The results were shown in FIG. 1D, and an average concentration of DEX in the Mφ-MP-DEX was about 6.2 μg/1×10¹⁰ MPs.

The results of the embodiment show that the dexamethasone-loaded macrophage-derived microvesicle prepared by the present invention could express macrophage marker proteins F4/80 and CD68 with the average diameter of 140.7±4.8 nm, the morphology observed under the transmission electron microscope conformed to the characteristics of the microvesicle, and the average concentration of DEX entrapped in the microvesicle is about 6.2 μg/1×10¹⁰ MPs.

### Second Embodiment

Therapeutic Effect of a Dexamethasone-Loaded Macrophage-Derived Microvesicle (Mφ-MP-DEX) on LPS-Induced Glomerular Endothelial Cell Inflammation

1. Detection of Mφ-MP-DEX Taken Up by a Glomerular Endothelial Cell

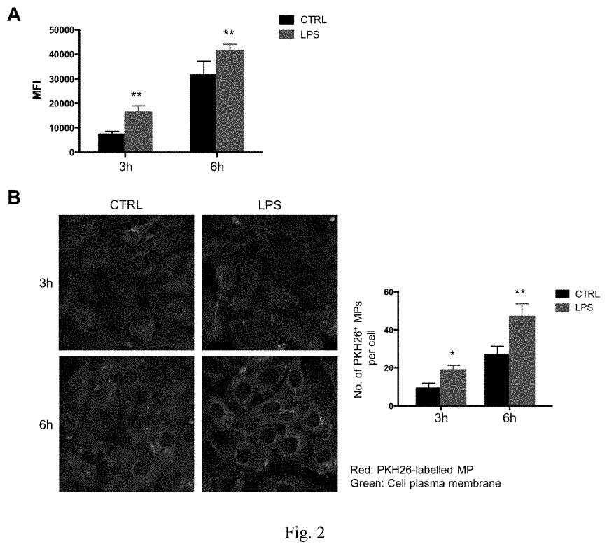

Primary glomerular endothelial cells (GECs) of a mouse were extracted in vitro and laid in a six-well plate or a confocal small dish, when the cell fusion reached 80%, 2×10⁷ Mφ-MP-DEXs labeled with PKH26 (Sigma of America) were added into the six-well plate, 5×10⁶ Mφ-MP-DEXs labeled with PKH26 were added into the confocal small dish, and effects of LPS (a final concentration of 10 μg/ml) (Sigma of America) on the uptake of Mφ-MP-DEX by GECs at different time points were observed; in order to observe the amount of Mφ-MP-DEX phagocytosed by GECs, flow cytometry was used to detect an average fluorescence intensity of GECs in the six-well plate, the results were shown in FIG. 2A, and the average fluorescence intensity of the GECs of an LPS-intervened group (a final concentration of 10 μg/ml) was higher (a control group was not added with LPS). An number of Mφ-MP-DEXs taken up by GEC was observed by a laser confocal microscope, the results were shown in FIG. 2B, and more Mφ-MP-DEXs were taken up by GECs of the LPS-intervened group.

2. Anti-Inflammatory Effect of Mφ-MP-DEX

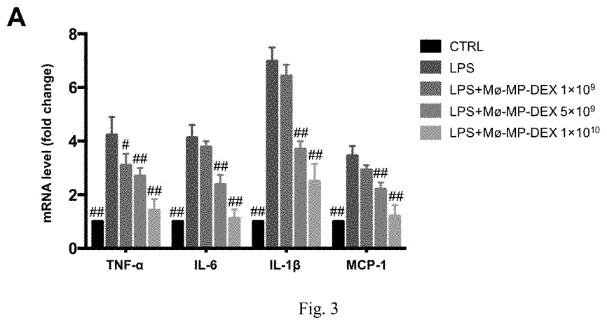

GECs were seeded in a six-well plate, when the cells reached 80% confluence, different doses of Mφ-MP-DEXs were added into each well, the doses were 1×10⁹, 5×10⁹ and 1×10¹⁰ Mφ-MP-DEXs respectively, and LPS (a final concentration of 15 μg/ml) was added to stimulate for 12 h to collect the cells. The mRNA levels of pro-inflammatory factors TNF-α, IL-6, IL-1β and MCP-1 were detected by RT-PCR. The results were shown in FIG. 3. With the increased dose of Mφ-MP-DEX, the expression of various pro-inflammatory factors gradually decreased.

3. Comparison of Anti-Inflammatory Effects Between Mφ-MP-DEX and DEX

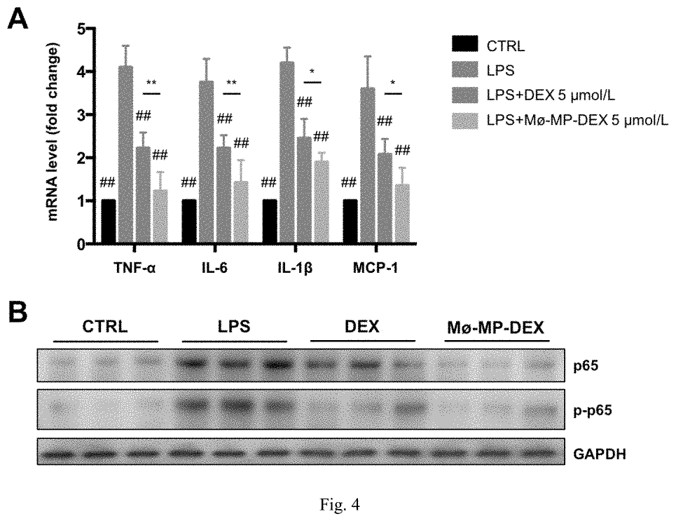

GECs were seeded in a six-well plate, when the cells reached 80% confluence, DEX and Mφ-MP-DEX with the same drug concentration (a final concentration of 5 μmol/L) were added respectively. Cells were collected 12 h after LPS (a final concentration of 15 μg/ml) stimulation. The mRNA level of pro-inflammatory factors TNF-α, IL-6, IL-1β and MCP-1 were detected by RT-PCR. The protein levels of NF-κB p65 and p-p65 were detected by Western blot. The results were shown in FIG. 4, and compared with the DEX group, Mφ-MP-DEX could better reduce the expression of the pro-inflammatory factor, and NF-κB p65 and p-p65.

The results of the embodiment show that the prepared Mφ-MP-DEX can be more easily taken up by inflamed GECs, and the anti-inflammatory effect thereof was dose-dependent; in comparison to direct DEX administration, the microvesicle prepared by the present invention had better anti-inflammatory effect.

### Third Embodiment

Therapeutic Effect of a Dexamethasone-Loaded Macrophage-Derived Microvesicle (Mφ-MP-DEX) on LPS-Induced Mouse Sepsis Model

C57BL/6 mice used in the experiment were 8 to 10 weeks old, and were purchased from Beijing Weitonglihua Experimental Animal Technology Co., Ltd. Experiment groups: control group: saline was injected intraperitoneally at 0 h and 24 h, and saline was injected intravenously at 12 h and 36 h; model group: LPS (10 μg/g) was injected intraperitoneally at 0 h and 24 h, and saline was injected intravenously at 12 h and 36 h; DEX treatment group: LPS (10 μg/g) was injected intraperitoneally at 0 h and 24 h, and DEX (DEX concentration was 0.5 mg/kg) was injected intravenously at 12 h and 36 h; and Mφ-MP-DEX treatment group: LPS (10 μg/g) was injected intraperitoneally at 0 h and 24 h, and Mφ-MP-DEX (DEX concentration was 0.5 mg/kg) was injected intravenously at 12 h and 36 h. All mice were sacrificed at 48 h.

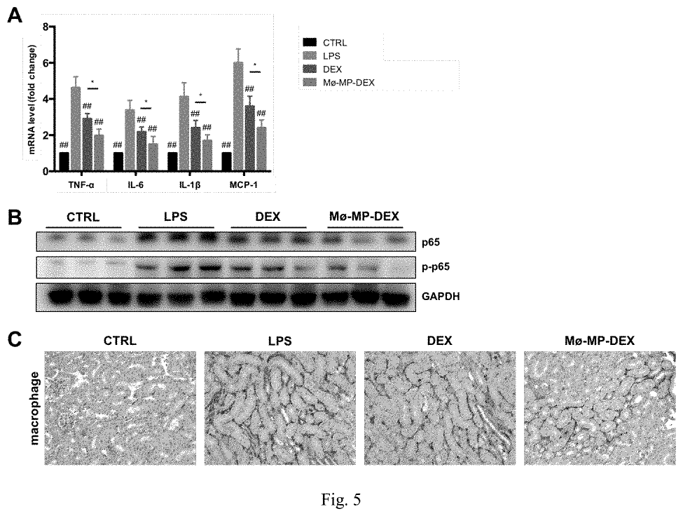

The mRNA levels of pro-inflammatory factors TNF-α, IL-6, IL-10 and MCP-1 in a kidney tissue were detected by RT-PCR, and the results were shown in FIG. 5A. Compared with the DEX group, the Mφ-MP-DEX could better reduce the expression of the pro-inflammatory factor.

The protein levels of NF-κB p65 and p-p65 were detected by Western blot, and the results were shown in FIG. 5B. Compared with the DEX group, the Mφ-MP-DEX could better reduce the protein expression of NF-κB p65 and p-p65.

The interstitial infiltration of the macrophage (F4/80⁺ cell) was detected by immunohistochemistry, and the results were shown in FIG. 5C. Compared with the DEX group, a number of the macrophages in the tubulointerstitium of the Mφ-MP-DEX group was fewer.

The results of the embodiment show that the therapeutic effect of the Mφ-MP-DEX on kidney inflammation in the mouse sepsis model is significantly better than that of direct DEX administration, which is mainly manifested in that the Mφ-MP-DEX is more effective than the DEX in inhibiting a pro-inflammatory signal pathway, reducing the expression of the pro-inflammatory factor, and reducing the interstitial infiltration of the macrophage.

Statistical Analysis

Statistical data is given in the form of average value±standard error, and processed by statistical software SPSS 13.0. One-way analysis of variance is used for comparison among groups, and t-test is used for comparison between two groups, wherein p<0.05 has significant difference. All the experimental results are repeated more than three times.

What is claimed is:

1. A dexamethasone-loaded macrophage-derived microvesicle, wherein the dexamethasone-loaded macrophage-derived microvesicle is formed by entrapping dexamethasone with a microvesicle derived from a murine macrophage cell line RAW264.7 cell.

2. A preparation method of the dexamethasone-loaded macrophage-derived microvesicle according to claim 1, comprising the following steps:
(1) culturing a murine macrophage cell line RAW264.7 cell;
(2) processing the RAW 264.7 cell with dexamethasone; and
(3) collecting medium supernatant after the cell is processed with dexamethasone in step (2), and collecting a microvesicle by differential centrifugation to obtain the dexamethasone-loaded macrophage-derived microvesicle.

3. The preparation method according to claim 2, wherein a specific process of culturing the RAW 264.7 cell of the mouse macrophage system in step (1) is: culturing the RAW 264.7 cell in a RIPM 1640 medium containing fetal bovine serum.

4. The preparation method according to claim 3, wherein the fetal bovine serum for culturing the RAW 264.7 cell is preferably a fetal bovine serum of which a microvesicle is removed.

5. The preparation method according to claim 2, wherein a specific process of processing the RAW 264.7 cell with dexamethasone in step (2) is: when the RAW 264.7 cell cultured in step (1) reaches 70% to 80% confluence, washing the cell with a RIPM 1640 medium not containing fetal bovine serum, and then replacing the medium by the RIPM 1640 medium without fetal bovine serum, and then adding dexamethasone to process.

6. The preparation method according to claim 2, wherein a specific process of step (3) is: collecting the medium supernatant after the cell is processed with dexamethasone in step (2), conducting low speed centrifugation firstly to obtain the supernatant, then conducting high speed centrifugation to discard the supernatant, adding PBS for resuspension washing, and then conducting high speed centrifugation to obtain a sediment which is the dexamethasone-loaded macrophage-derived microvesicle.

7. The dexamethasone-loaded macrophage-derived microvesicle according claim 1, the dexamethasone-loaded macrophage-derived microvesicle is preparing for a drug or for treating kidney diseases.

8. The dexamethasone-loaded macrophage-derived microvesicle according to claim 7, wherein the dexamethasone-loaded macrophage-derived microvesicle can be better taken up by an injured cell and inhibit the activation of a proinflammatory signal pathway and infiltration of inflammatory cells.

9. The dexamethasone-loaded macrophage-derived microvesicle according claim 1, the dexamethasone-loaded macrophage-derived microvesicle is prepared as an anti-inflammatory or immunosuppressive drug.
